(19) United States
(12) Patent Application Publication
(10) Pub. No.: US 2008/0312250 A1
(43) Pub. Date: Dec. 18, 2008

(54) Combination Comprising a Bcrp Inhibitor and 4- (4-Methylpiperazin-1-Ylmethyl)-N-[4-Methyl-3- (4-Pyridin-3-Yl) Pyrimidin-2-Ylamino) Phenyl] -Benzamide

(75) Inventors: Pauline Breedveld, Weesp (NL); Greta Cipriani, Pistoia (IT); Dick Pluim, Amsterdam (NL); Johannes Henricus Matthias Schellens, Kockengen (NL); Olaf Van Tellingen, Zaandam (NL); Pieter Roeland Wielinga, Amsterdam (NL)

Correspondence Address: NOVARTIS;CORPORATE INTELLECTUAL PROPERTY, ONE HEALTH PLAZA 104/3, EAST HANOVER, NJ, 07936-1080, US

(73) Assignee: THE NETHERLANDS CANCER INSTITUTE, Amsterdam (NL)

(21) Appl. No.: 11/570,888

(22) PCT Filed: Jun. 30, 2005
(86) PCT No.: PCT/EP2005/007090

§ 371 Date: Aug. 19, 2008

(51) Int. Cl. A61K 31/506; A61P 35/04

(52) U.S. Cl. 514/252.18

(57) ABSTRACT

The present invention relates to a combination which comprises a BCRP inhibitor and Compound I of formula (I),

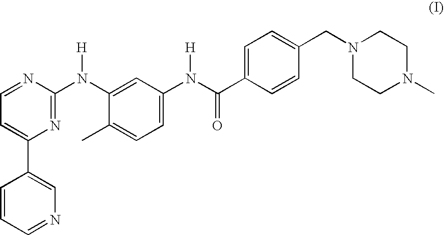

in which the active ingredients are present in each case in free form or in the form of a pharmaceutically acceptable salt and optionally at least one pharmaceutically acceptable carrier for simultaneous, separate or sequential use, especially in the delay of progression or treatment of cancer, and to pharmaceutical compositions comprising such combinations.

The invention relates to a combination which comprises (a) a BCRP inhibitor and (b) Compound I or pharmaceutically acceptable salts thereof, for simultaneous, separate or sequential use in the treatment of diseases, or delay of progression of diseases, in particular cancer, especially cancers which are pharmacoresistant, especially resistant to Compound I; the use of such combination for the preparation of a medicament for such delay of progression or treatment of cancer; and to a method of prevention, delay of progression or treatment of cancer.

The invention relates to a combination which comprises (a) a BCRP inhibitor, (b) Compound I, and (c) a P-gp inhibitor or pharmaceutically acceptable salts thereof, for simultaneous, separate or sequential use in the treatment of diseases, or delay of progression of diseases, in particular cancer.

Compound I is 4-(4-methylpiperazin-1-ylmethyl)-N-[4-methyl-3-(4-pyridin-3-yl)pyrimidin-2-ylamino)phenyl]-benzamide having the following formula I

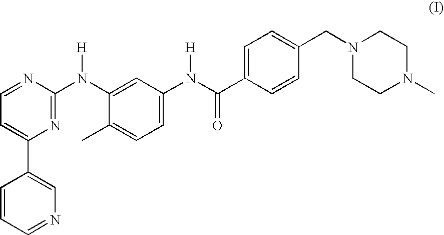

Compound I free base, its acceptable salts thereof and its preparation are disclosed in the European granted patent 0564409. Compound I free base corresponds to the active moiety.

The monomethanesulfonic acid addition salt of Compound I and a preferred crystal form thereof, e.g. the beta crystal form, are described in PCT patent application WO99/03854 published on Jan. 28, 1999.

Compound I has demonstrated marked clinical efficacy and safety in Bcr/Abl-expressing chronic myeloid leukaemia and c-Kit-expressing gastro-intestinal stromal tumors. Compound I or a pharmaceutically acceptable salt thereof is a potent and selective tyrosine kinase inhibitor, which has been shown to effectively inhibit Platelet-derived Growth Factor (PDGF)-induced glioblastoma cell growth preclinically.

Primary tumors of the central nervous system, e.g. glioblastoma multiform and anaplastic astrocytoma, are respectively the third and fourth leading cause of cancer-related death among male and female young adults. Moreover, primary brain tumors are the most common solid tumor of childhood and the second leading cause of cancer death in children after leukaemia. Recently, central nervous system (CNS) tumor relapses and leukaemia have been reported in CML patients receiving chronic p.o. Compound I after successful attainment and maintenance of systemic remission. Unfortunately, the treatment of CNS tumors is often limited by low distribution of antitumor agents into the brain as a result of a proficient blood-brain barrier containing various efflux transporters. These include P-glycoprotein (MDR1, ABCB1) and Breast Cancer Resistance Protein (ABCG2), which can eliminate xenobiotics from the brain against a concentration gradient, thereby limiting CNS exposure to these compounds. A limited penetration of Compound I into the cerebrospinal fluid of humans and nonhuman primates has been reported.

Surprisingly, it has now been found that the administration of a combination disclosed herein results in an increased local concentration of the drug. Such finding qualifies the combinations disclosed herein to be more suitable to treat cancer, e.g. which is resistant to antineoplastic drugs, than the corresponding antineoplastic drug alone.

The present invention relates to a combination, such as a combined preparation or pharmaceutical composition, which comprises a BCRP inhibitor and Compound I or pharmaceutical acceptable salt thereof, in which the active ingredients are present in each case in free form or in the form of a pharmaceutically acceptable salt and optionally at least one pharmaceutically acceptable carrier; for simultaneous, separate or sequential use, particularly, in the delay of progression or treatment of cancers, in particular in brain cancer, especially glioma cancers. Such a combination is preferably a combined preparation or a pharmaceutical composition.

By the term “a combined preparation or pharmaceutical composition for simultaneous, separate or sequential use”, there is meant especially a “kit of parts” in the sense that the components, BCRP inhibitor and Compound I can be dosed independently or by use of different fixed combinations with distinguished amounts of the components, i.e. at different time points or simultaneously. The parts of the kit of parts can then e.g. be administered simultaneously or chronologically staggered, that is at different time points and with equal or different time intervals for any part of the kit of parts. Preferably, the time intervals are chosen such that the effect on the treated disease or condition in the combined use of the parts is larger than the effect which would be obtained by use of only any one of the components.

The term “delay of progression” used herein means that the administration of the combination to patients will result in a slower or no progression of the disease, than if the patients would not have been treated.

The term “pharmacoresistant” or “pharmacoresistance” as used herein in conjunction with cancer relates to a cancer which is or becomes refractory to the treatment with an drug, e.g. applied in a dosage and during a term which constitute about the standard regimen for said drugs. For example pharmacoresistant cancer can be, e.g. refractory to Compound I treatment or e.g. where Compound I is becoming less or not efficient against said cancer, e.g. where Compound I, becomes less bioavailable, e.g. due to active drug efflux from the cells.

By “cancer” is meant but without limitation for example liquid and solid tumors, e.g. chronic myelogenous leukemias CML, acute lymphoblastic leukemias (ALL), gastro-intestinal stromal tumors (GIST), brain cancer, e.g. primary tumors of the central nervous system, e.g. gliomas, glioblastoma multiforme, anaplastic astrocytoma.

The term “BCRP inhibitor” as used herein relates to compounds which inhibit the activity of the breast cancer resistant protein. The term includes, but is not limited to, pantroprazole, the Aspergillus fumigatus secondary metabolite tryprostatin A, fumitremorgin C abbreviated as FTC and its derivatives the demethoxy-fumitremorgin C analogs, Ko132, Ko134, Ko143, GF120918, the quinazoline-based HER family tyrosine kinase inhibitor CI1033, estrogens like estrone and 17beta-estradiol, e.g. estradiol-17-beta-D-glucuronide.

GF120918, called Elacridar can be obtained from GlaxoWellcome, Research Triangle Park, N.C.

Pantoprazole, Pantozol®, is available from Altana Pharma, Hoofddorp, The Netherlands.

The term “P-gp inhibitor” as used herein relates to compounds which inhibit the activity of the P-glycoprotein (P-gp). The term P-gp inhibitor includes but is not limited to verapamil, [3′-desoxy-3′-oxo-MeBmt]¹-Ciclosporin, [3′-desoxy-3′-oxo-MeBmt]¹-[Val]²-Ciclosporin and [3′-desoxy-3′-oxo-MeBmt]¹-[Nva]²-Ciclosporin disclosed in EP 0 296 122 in Example H as cyclosporins 1.37, 1.38 and 1.39, respectively, as well as Cyclo-[Pec-MeVal-Val-MeAsp(β-P-t-Bu)-MeIle-MeIle-Gly-MeVal-Tyr(Me)-L-Lact] and Cyclo-[Pec-MeVal-Val-MeAsp-MeIle-MeIle-Gly-MeVal-Tyr(Me)-D-Lact], disclosed in EP. 0 360 760 as Examples 52 and 1 (first compound), respectively. With regard to all aspects of the present invention, preferably [3′-desoxy-3′-oxo-MeBmt]¹-[Val]²-Ciclosporin A, also known as valspodar, hereinafter referred to as PSC833, known from EP 0 296 122 (Example H) is used as the P-gp inhibitor. PSC833 can be administered in the form of the galenical composition disclosed in WO 93/20833.

The active ingredients or a pharmaceutically acceptable salt thereof may also be used in form of a hydrate or include other solvents used for crystallization.

The structure of the active agents identified by code numbers, generic or trade names may be taken from the actual edition of the standard compendium “The Merck Index” or from databases, e.g. Patents International (e.g. IMS World Publications). The corresponding content thereof is hereby incorporated by reference.

It can be shown by established test models and especially the test model described herein that the combination of a BCRP inhibitor, and Compound I or in each case a pharmaceutically acceptable salt thereof, results in a more effective delay of progression or treatment of cancer. The pharmacological activity may, for example, be demonstrated following essentially the in-vivo test procedure in rats or in a clinical study as described hereinafter.

Furthermore, the present invention relates to a combined preparation which comprises a BCRP inhibitor and Compound I in which the active ingredients are present in each case in free form or in the form of a pharmaceutically acceptable salt and optionally at least one pharmaceutically acceptable carrier, as a combined preparation for simultaneous, separate or sequential use.

It is one objective of this invention to provide a pharmaceutical composition comprising an amount of (i) a BCRP inhibitor and (ii) Compound I or a pharmaceutically acceptable salt thereof, amount which is jointly therapeutically effective in the treatment of cancer, especially pharmacoresistant cancers, and at least one pharmaceutically acceptable carrier. In this composition, the components (i) and (ii) can be administered together, one after the other or separately in one combined unit dosage form or in two separate unit dosage forms. The unit dosage form may also be a fixed combination.

The pharmaceutical compositions according to the invention can be prepared in a manner known per se and are those suitable for enteral, such as oral or rectal, and parenteral administration to mammals (warm-blooded animals), including man, comprising a therapeutically effective amount of the pharmacologically active compound, alone or in combination with one or more pharmaceutically acceptable carriers, especially suitable for enteral or parenteral application.

The novel pharmaceutical preparations contain, for example, from about 10% to about 100%, preferably 80%, preferably from about 20% to about 60%, of the active ingredient. Pharmaceutical preparations for the combination therapy that may be used for enteral or parenteral administration are, for example, those in unit dose forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. Thus, pharmaceutical preparations for oral use can be obtained by combining the active ingredient with solid carriers, if desired granulating a mixture obtained, and processing the mixture or granules, if desired or necessary, after addition of suitable excipients to give tablets or sugar-coated tablet cores.

It will be appreciated that the unit content of active ingredient or ingredients contained in an individual dose of each dosage form need not in itself constitute an effective amount since the necessary effective amount can be reached by administration of a plurality of dosage units.

In particular, a therapeutically effective amount of each of the components of the combination of the present invention may be administered simultaneously or sequentially and in any order, and the components may be administered separately or as a fixed combination. The individual components of the combination can be administered separately at different times during the course of therapy or concurrently in divided or single combination forms. Furthermore, the term administering also encompasses the use of prodrugs of any of the drugs that convert in vivo to the selective drugs. The instant invention is therefore to be understood as embracing all such regimes of simultaneous or alternating treatment and the term “administering” is to be interpreted accordingly.

The preferred route of administration of the dosage forms of the present invention is enterally or, preferably, orally. Because of their ease of administration, tablets and capsules represent the most advantageous oral dosage unit form in which case solid pharmaceutical carriers are obviously employed.

The effective dosage of each of the active ingredients employed in the combination therapy may vary depending on the particular pharmaceutical composition employed, the mode of administration, or the severity of the condition being treated. A physician, clinician or veterinarian of ordinary skill can readily determine and prescribe the effective amount of the drug required to prevent, counter or arrest the progress of the condition.

In accordance with the present invention there is further provided a method of prevention, delay of progression or treatment of and a pharmaceutical composition for the delay of progression or treatment of cancer, especially cancer which is resistant to antineoplastic drug, e.g. resistant to Compound I. The treatment involves administering to a patient in need of such treatment a pharmaceutical composition comprising a pharmaceutical carrier and a therapeutically effective amount of each compound in the combination of the present invention.

In one embodiment of the invention a combination as disclosed herein is administered locally to the brain of a mammal, especially a human, suffering from cancer or another disease mentioned herein. Such a local administration can, e.g., be accomplished by means of a small pump placed under the skin of the mammal, which pump, e.g. continuously, provides such combination to a particular region of the body, e.g. of the brain. Hence, the present invention pertains also to the use of a combination as disclosed herein for the preparation of a medicament wherein the medicament is adapted for local administration to a particular region of the brain of a mammal.

The invention relates in particular to a commercial package comprising jointly therapeutically effective amounts of a BCRP inhibitor and Compound I, in free or pharmaceutically acceptable salt form in each case, together with instructions for use thereof in the treatment of cancer, especially cancer which is resistant to antineoplastic drugs.

### EXAMPLE 1

Materials. Pantoprazole (Pantozol. 40 mg i.v., Altana Pharma, Hoofddorp, The Netherlands) is obtained from the pharmacy of the Netherlands Cancer Institute. Elacridar (GF120918) is a generous gift from Glaxo Wellcome (Research Triangle Park, N.C.). In this example “Compound I” refers to Compound I mesylate

Transport across MDCKII monolayers. The MDCKII (Madin-Darby canine kidney strain II) cells are cultured in Dulbecco's Modified Eagle's Medium supplemented with 10% fetal calf serum and 100 units penicillin/streptomycin per ml. Cells are grown at 37° C. with 5% CO2 under humidifying conditions. Polarized MDCKII cells stably expressing human MRP2 (ABCC2), or murine Bcrp1 (Abcg1) cDNA have been described before (Jonker et al., J. Natl. Cancer. Inst. 2000, 92:1651-56). Transepithelial transport assays are performed as described previously (Jonker et al., J. Natl. Cancer. Inst. 2000, 92:1651-56). Animals. Animals used in this study are male Bcrp1−/− (Bcrp1 knockout), Mdr1a/1b−/− (Mdr1a/1b knockout) and wild type mice of a comparable genetic background (FVB) between 9 and 14 weeks of age. Mice are housed and handled according to institutional guidelines complying with Dutch legislation.

Drug solutions. A mixture of Compound I and [¹⁴C]Compound I (approximately 3 μCi) is diluted with NaCl 0.9% to a final concentration of 1.6 mg/ml. A vial of pantoprazole (Pantozol® 40 mg) is diluted with NaCl 0.9% to a final concentration of 8 mg/ml. Elacridar is suspended at 10 mg/ml in a mixture of hydroxypropylmethylcellulose (10 g/L)/2% Tween 80/H2O (0.5:1:98.5 [vol/vol/vol] for oral administration).

Drug administration and analysis. All mice receive [¹⁴C] Compound I by intravenous administration in the tail vein at a dose of 12.5 mg/kg. The study comprised 7 different study groups:

1. Wild type control mice, receiving i.v. NaCl 0.9% 3 min prior to Compound I;
2. Bcrp1 knockout mice, receiving i.v. NaCl 0.9% 3 min prior to Compound I;
3. Mdr1a/1b knockout mice, receiving i.v. NaCl 0.9% 3 min prior to Compound I;
4. Wild type mice, receiving p.o. elacridar (100 mg/kg) (19) 2 h prior to Compound I;
5. Wild type mice, receiving i.v. pantoprazole (40 mg/kg) (15) 3 min prior to Compound I;
6. Bcrp1 knockout mice, receiving i.v. pantoprazole (40 mg/kg) (15) 3 min prior to Compound I;
7. Mdr1a/1b knockout mice, receiving i.v. pantoprazole (40 mg/kg) (15) 3 min prior to Compound I;

Blood samples (30 μl) are taken from the tail vein at 5, 15, 30, 60, 90 and 120 min after Compound I administration. After the last sampling time-point animals are anaesthetized with methoxyflurane, their remaining blood collected by cardiac puncture and organs are removed after sacrifice by cervical dislocation. Coagulation of blood is prevented by use of heparinized capillaries for blood sampling. The plasma fraction of the blood samples is collected after centrifugation at 3000 g for 5 min. The organs are homogenized in 4% (wt/vol) BSA. Radioactivity in the plasma samples and the tissue homogenates is determined by liquid scintillation counting (Tri-Carb. 2100 CA Liquid Scintillation analyzer, Canberra Packard, Groningen, The Netherlands).

Pharmacokinetic and statistical analysis. Pharmacokinetic parameters after i.v. administration of Compound I are calculated by noncompartmental methods using the software package MW\Pharm (MED\WARE, version 3.02). The area under the plasma concentration-time curve is calculated from 0 to 120 minutes using the linear-logarithmic trapezoidal method. The clearance is calculated by the formula Cl= dose/AUC. The two-sided unpaired Student's t test is used to assess the statistical significance of difference between two sets, of data. Results are presented as means±standard deviation (SD). Differences are considered to be statistically significant when p<0.05.

### Results and Discussion

Transport of Compound I across MDCKII monolayers. Transport of Compound I by Bcrp1 is studied in MDCKII-Bcrp1 and MDCKII parental cells (15). To exclude any contribution of P-gp (12), the P-gp inhibitor zosuquidar (5 μM) is added (18). Efficient transport of 1 and 10 μM Compound I by Bcrp1 (approximately 20% net active transport per h), which is saturable at concentrations above 10 μM. Compound I is not transported by MRP2 (data not shown).

Effect of pantoprazole and elacridar on Bcrp1-mediated transport of Compound I in vitro. The effect of pantoprazole and elacridar on the transport of 1 μM Compound I is also investigated in MDCKII transfected cells. In the experiments in which the effect of pantoprazole is studied, the P-gp inhibitor zosuquidar (5 μM) is added to exclude any contribution of P-gp. Pantoprazole and elacridar inhibit the Bcrp1-mediated transport of Compound I (data not shown).

Role of Bcrp1 in the clearance of Compound I in mice. [C] Compound I (12.5 mg/kg) is administered to Bcrp1−/− (Bcrp1 knockout), Mdr1a/1b−/− (P-gp knockout) and wild type mice, and the clearance after measurement of Compound I plasma, concentrations by total radioactivity over a 120 min time period is determined. The clearance of i.v. Compound I is 1.6-fold decreased in Bcrp1 knockout mice compared to control mice (p<0.01). In P-gp knockout mice the clearance of i.v. Compound I is 1.25-fold decreased compared to control mice (p<0.01). These results show that Bcrp1 plays an important, and maybe even a more prominent role than P-gp, in the clearance of i.v. Compound I in mice.

Effect of P-gp and Bcrp1 inhibitors on the clearance of Compound I in mice, i.v. [14C] Compound I (12.5 mg/kg) is administered to mice, which are pretreated either with elacridar, or with pantoprazole, or with solvent only as control. The clearance of i.v. Compound I in wild type mice pretreated with elacridar is 1.5-fold decreased compared to control mice (p<0.05) and is not significantly different from the clearance in Bcrp1 knockout and P-gp knockout mice (data not shown). The clearance of i.v. Compound I in mice pretreated with pantoprazole is 1.7-fold decreased compared to control mice (p<0.001). In Bcrp1 knockout mice co-administered with pantoprazole the clearance of i.v. Compound I is 1.7-fold decreased compared to control wild type mice (p<0.001) and is not significantly different from control Bcrp1 knockout mice. In P-gp knockout mice pretreated with pantoprazole the clearance of i.v. Compound I is 1.7-fold decreased compared to control mice (p<0.001) and is 1.4-fold decreased compared to control P-gp knockout mice (p<0.001) (data not shown). These results suggest that administration of pantoprazole with Compound I decreases the clearance of i.v. Compound I by competition for Bcrp1. Overall, these data show that co-administration of a P-gp and BCRP inhibitor reduces the clearance of i.v. Compound I, in line with the results obtained with the knockout mice. Effect of Bcrp1 on the brain penetration of Compound I in mice. The brain concentration of Compound I is determined by measuring the radioactivity in whole brain homogenates, which are collected 2 h after administration of i.v. Compound I. Because Compound I has a low CNS distribution, the brain concentration of Compound I in the brain vascular space (i.e. 1.4% of the plasma concentration at t= 2 h) is subtracted from the brain concentration found in whole brain homogenates. The brain penetration of Compound I is calculated by determining the Compound I brain concentration at t= 2 h relative to the plasma AUC(0-2h), as the AUC better reflects the overall Compound I exposure to the brain than the plasma concentration at 2 h after administration. As shown in FIG. 4, the brain penetration of Compound I in Bcrp1 knockout mice is 2.5-fold increased compared to control mice (p<0.01), whereas in P-gp knockout mice this is 3.6-fold increased (p<0.01). These results show that the presence of Bcrp1 in the blood-brain barrier limits the brain penetration of Compound I, but to a lower extent than P-gp. Effect of P-gp and Bcrp1 inhibitors on the brain penetration of Compound I in mice. As shown in FIG. 4, co-administration of the P-gp and BCRP inhibitor elacridar in wild type mice increased the relative brain penetration of Compound 14.2-fold compared to control mice (p<0.05), 1.7-fold compared to Bcrp1 knockout mice (p=0.08), and 1.2-fold compared to P-gp knockout mice (p=0.45). Taken into account that P-gp inhibition with a single dose of elacridar is approximately 70-80% (19), the role for Bcrp1 in the brain penetration of Compound I is likely more important than suggested by the 1.2-fold increase in P-gp knockout mice. Thus, co-administration of elacridar effectively increases the brain penetration of Compound I, by inhibition of both P-gp and Bcrp1 at the blood-brain barrier. The brain penetration of Compound I in wild type mice treated with pantoprazole is 1.8-fold increased compared to control mice (p<0.05) (FIG. 1). In P-gp knockout mice treated with pantoprazole the Compound I brain penetration is 4.7-fold increased compared to control wild type mice (p<0.01) and 1.3-fold compared to control P-gp knockout mice (p<0.05). Thus, when P-gp is absent, additional inhibition of Bcrp1 by pantoprazole increases the brain penetration of Compound I even more. In Bcrp1 knockout mice treated with pantoprazole the brain penetration of Compound I increased 2.3-fold compared to control mice (p<0.05) and is not significantly different from control Bcrp1 knockout mice. These results suggest that administration of pantoprazole increases the brain penetration of Compound I in mice by inhibition of Bcrp1 and not by P-gp inhibition. This is in line with the results showing that pantoprazole inhibits the Bcrp1-mediated clearance of Compound I. In conclusion, our results show that besides P-gp also Bcrp1 plays an important role in the pharmacokinetics and brain penetration of Compound I. The brain penetration of Compound I can be improved by the co-administration of P-gp and/or BCRP inhibitors, like elacridar and pantoprazole. The results suggest that inhibition of both Bcrp1 and P-gp is possibly more effective than inhibition of P-gp alone to increase the brain penetration of Compound I. As a recent report about the first multicenter phase II study of Compound I mesylate in patients with recurrent glioblastoma showed promising antitumor activity, co-administration of BCRP and P-gp inhibitors may improve the delivery of Compound I mesylate to malignant gliomas. Therefore, clinical trials with oral Compound I combined with Bcrp1 and Pgp inhibitors are warranted.

### FIGURE LEGEND

FIG. 1. Brain penetration of [¹⁴C]Compound 1 (12.5 mg/kg) in mice.

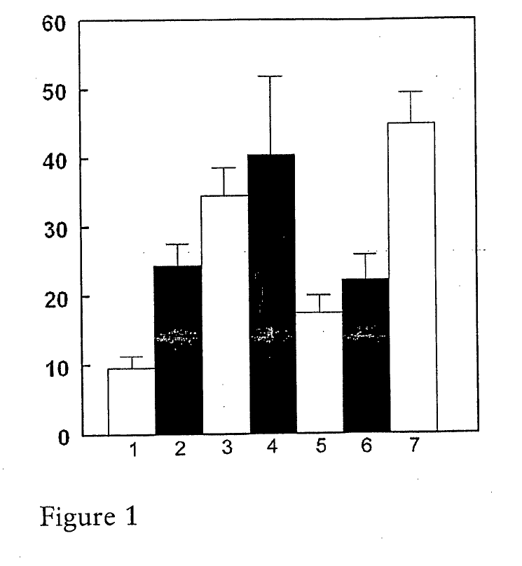

Control wild type mice (WT) are treated with i.v. NaCl 0.9% 3 minutes prior to an i.v. dose of [14C]Compound I. Bcrp1 knockout (k.o.) and P-gp knockout (k.o.) mice are pretreated with i.v. NaCl 0.9% (control) and compared with control mice to determine the role of Bcrp1 relative to P-gp in the brain penetration of Compound I. WT mice are treated with p.o. elacridar (GF120918) (100 mg/kg) 2 h prior to an i.v. dose of [¹⁴C] Compound I and compared with control WT mice and with control Bcrp1 k.o. and control Mdr1a/1b k.o. mice to determine the effect of a P-gp and BCRP inhibitor on the brain penetration of Compound I. WT, Bcrp1 k.o. and Mdr1a/1b k.o. mice are treated with i.v. pantoprazole (40 mg/kg 120 mg/m2) 3 minutes prior to an i.v. dose of [¹⁴C]Compound I and compared with control to determine the effect of the proton pump inhibitor pantoprazole on the brain penetration of Compound I. The above-mentioned mice groups are referred by the number 1 to 7 below the columns on FIG. 1: 1. Wild type control mice, receiving i.v. NaCl 0.9% 3 min prior to Compound I; 2. Bcrp1 knockout mice, receiving i.v. NaCl 0.9% 3 min prior to Compound I; 3. Mdr1a/1b knockout mice, receiving i.v. NaCl 0.9% 3 min prior to Compound I; 4. Wild type mice, receiving p.o. elacridar (100 mg/kg) (19) 2 h prior to Compound I; 5. Wild type mice, receiving i.v. pantoprazole (40 mg/kg) (15) 3 min prior to Compound I; 6. Bcrp1 knockout mice, receiving i.v. pantoprazole (40 mg/kg) (15) 3 min prior to Compound I; 7. Mdr1a/1b knockout mice, receiving i.v. pantoprazole (40 mg/kg) (15) 3 min prior to Compound I. The y-axis provides Compound I brain penetration ×10⁻³ h⁻¹. At 2 h post-dose the plasma and whole brain tissue homogenate are collected and counted for radioactivity. The brain penetration, calculated as the brain concentration at t= 2 h to plasma AUC(0-2h) ratio of each test group, is plotted (the brain concentration is corrected for the brain vascular space, i.e. 1.4% of plasma concentration at t= 2 h). Results are expressed as mean±SD (n=3).

1. Combination which comprises a BCRP inhibitor and Compound I of formula I 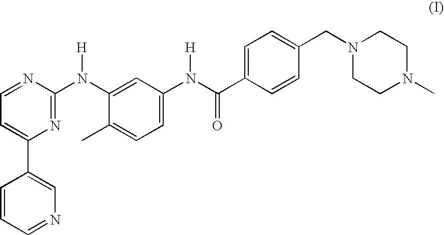
in which the active ingredients are present in each case in free form or in the form of a pharmaceutically acceptable salt and optionally at least one pharmaceutically acceptable carrier; for simultaneous, separate or sequential use, with the proviso that the BCRP inhibitor is not Compound I.

2. Combination according to claim 1 which is a fixed pharmaceutical composition.

3. Combination according to claim 1 wherein the BCRP inhibitor is selected from the group comprising pantroprazole, tryprostatin A, fumitremorgin C, Kol32, Kol34, Kol43, GF120918, the quinazoline-based HER family tyrosine kinase inhibitor CI1033, and estrogens.

4. Combination according to claim 3, wherein the BCRP inhibitor is GF120918.

5. Combination according to claim 3, wherein the BCRP inhibitor is pantroprazole.

6. Combination according to claim 1 for simultaneous, separate or sequential use in the delay of progression or treatment of cancer.

7. Method of treatment of a warm-blooded animal having cancer comprising administering to the animal a combination of (a) a BCRP inhibitor selected from the group comprising pantroprazole, tryprostatin A, fumitremorgin C, Kol32, Kol34, Kol43, GF120918, the quinazoline-based HER family tyrosine kinase inhibitor CI1033, and an estrogen and (b) Compound I or a pharmaceutically acceptable salt thereof in a quantity which is jointly therapeutically effective against cancer in which the compounds can also be present in the form of their pharmaceutically acceptable salts.

8. A pharmaceutical composition comprising a combination according to claim 1 in a quantity which is therapeutically effective against cancer and at least one pharmaceutically acceptable carrier.

9. A pharmaceutical composition according to claim 6 comprising a quantity, which is jointly therapeutically effective against cancer which pharmacoresistant, of a combination according to claim 1, and at least one pharmaceutically acceptable carrier.

10. (canceled)

11. A method according to claim 7, characterized in that the cancer is pharmacoresistant.

12. A method according to claim 7, wherein the cancer is a cancer selected from the group comprising CML, ALL, GIST, and brain cancers.

13. A method according to claim 12, wherein the brain cancer is a glioma.

14. Use A method according to claim 12, wherein the medicament is adapted for local administration to a particular region of the brain of a mammal.

15. A commercial package comprising as active agent (a) a BCRP inhibitor and 1b) Compound of formula I or a pharmaceutically acceptable salt thereof, together with instructions for simultaneous, separate or sequential use thereof in the delay of progression or treatment of cancer, with the proviso that (a) the BCRP inhibitor is not Compound I.

16. The combination according to claim 1 which further comprises a P-gp inhibitor.
